(19) United States
(12) Patent Application Publication
(10) Pub. No.: US 2019/0240369 A1
(43) Pub. Date: Aug. 8, 2019

(54) Ultraviolet Sterilization Apparatus

(71) Applicant: Cecilia Rotelli, Colorado Springs, CO (US)

(72) Inventor: Cecilia Rotelli, Colorado Springs, CO (US)

(21) Appl. No.: 16/257,144

(22) Filed: Jan. 25, 2019

Related U.S. Application Data
(60) Provisional application No. 62/626,926, filed on Feb. 6, 2018.

(51) Int. Cl. A61L 9/20; A61L 2/10

(52) U.S. Cl. CPC A61L 9/20; A61L 2202/20; A61L 2202/16; A61L 2/10

(57) ABSTRACT

An ultraviolet sterilization apparatus. The apparatus comprises a housing. The housing comprises a base disposed opposite of a top with at least one sidewall disposed between the base and the top. The housing defines an interior cavity. At least one light is disposed in the interior cavity of the housing. An opening is disposed through the housing into the interior cavity. The light is configured to emit ultraviolet radiation, such that the ultraviolet radiation will sanitize a surface that it comes into contact with. The light is in operable connection with an actuator.

### CROSS REFERENCE TO RELATED APPLICATIONS

This application claims the benefit of U.S. Provisional Application No. 62/626,926 filed on Feb. 6, 2018. The above identified patent application is herein incorporated by reference in its entirety to provide continuity of disclosure.

### BACKGROUND OF THE INVENTION

The present invention relates to an ultraviolet sterilization apparatus. More specifically, an ultraviolet sterilization apparatus is provided that is both versatile and user-actuatable, such that equipment can be sterilized at the convenience of the user.

Public equipment, including ATMs, credit card terminals, public restrooms, and other high traffic facilities are prone to becoming overexposed to germs, such as bacteria. Commonly, these germs will cause illnesses or ailments to individuals or customers that utilize these facilities. Though businesses or homeowners may seek to regularly clean their restrooms, germs can perambulate upon public equipment quickly and without notice. Some public buildings, such as hospitals, have an extremely strong interest in eliminating germs due to the vulnerability of patients or other individuals in those buildings.

Some individuals may carry sanitization means with them. For example, some individuals y carry sanitizing wipes or small bottles of sanitizing solution. However, these methods are not over effective as few people are willing to wipe down every surface, such as doorknobs, faucet handles and keypads. Furthermore, it can be difficult for many large rooms, such as school classrooms and hospital rooms to be cleaned on a daily basis. Additionally, sanitizing solutions may become ineffective without the user being aware of the ineffectiveness. As such, a means is desired for cleaning an area that is safe for a user, effective in eliminating pathogens and efficient in both cost and time.

### SUMMARY OF THE INVENTION

In view of the foregoing disadvantages inherent in the known types of area-directable sterilization apparatuses and devices now present in the known art, the present invention provides an ultraviolet sterilization apparatus wherein the same can be utilized for providing convenience for the user when sterilizing an area.

The present system comprises a housing. The housing comprises a base disposed opposite of a top with at least one sidewall disposed between the base and the top. The housing defines an interior cavity. At least one light is disposed in the interior cavity of the housing. An opening is disposed through the housing into the interior cavity. The light is configured to emit ultraviolet radiation, such that the ultraviolet radiation will sanitize a surface that it comes into contact with. The light is in operable connection with an actuator.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an ultraviolet sterilization apparatus.

FIG. 2 shows a perspective view of an alternate embodiment of the ultraviolet sterilization apparatus.

FIG. 3 shows a perspective view of an alternate embodiment of the ultraviolet sterilization apparatus.

FIG. 4 shows a perspective view of an alternate embodiment of the ultraviolet sterilization apparatus.

FIG. 5A shows a front perspective view of an alternate embodiment of the ultraviolet sterilization apparatus.

FIG. 5B shows a rear perspective view of an alternate embodiment of the ultraviolet sterilization apparatus.

### DETAILED DESCRIPTION OF THE INVENTION

Reference is made herein to the attached drawings. Like reference numerals are used throughout the drawings to depict like or similar elements of the ultraviolet sterilization apparatus. The figures are intended for representative purposes only and should not be considered to be limiting in any respect.

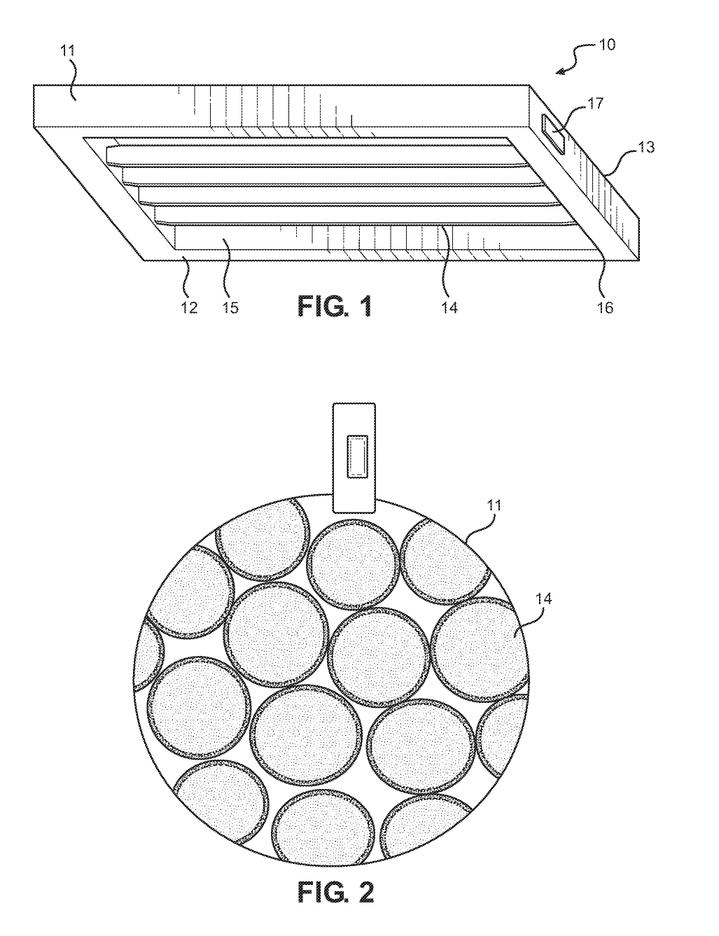

Referring now to FIG. 1, there is shown a perspective view of an ultraviolet sterilization apparatus. The ultraviolet sterilization apparatus 10 comprises a housing 11 having a base 12 disposed oppositely a top 13 with at least one sidewall disposed therebetween such that the housing 11 is defined. The housing 11 defines an interior cavity 15. In one embodiment, the housing 11 is constructed from a rigid material or a resilient material, such as a metal. In the illustrated embodiment, the housing 11 is rectangular, such that a linear length of the housing 11 is greater than a linear height of the housing 11. In alternate embodiments, however, the housing 11 can be of any suitable configuration for sanitizing an area or an object.

At least one light 14 is disposed in the interior cavity 15 of the housing 11. An opening 16 is defined in the housing 11, such that a beam of light may pass from the light 14 to the area surrounding the housing 11. The light 14 is configured to emit ultraviolet radiation, such that objects exposed to the ultraviolet radiation will be sanitized. In one embodiment, the light 14 is a mercury-vapor lamp. When utilizing a mercury-based lamp, UV is emitted at 253.7 nm. In another embodiment, the light 14 is a light-emitting diode. When utilizing a light emitting diode, the wavelength of the ultraviolet light is selectable between 255 nm and 280 nm.

The light 14 is in operable connection with an actuator 17. In the illustrated embodiment, the actuator 17 is a switch configured to be freely movable between an active position and an inactive position. The active position corresponds to the actuation of the light 14. The inactive position corresponds to the deactivation of the light 14. In one embodiment, the actuator 17 is placed in circuit between a power source and the light 14 such that the electrical current between the power source and the light 14 is engaged and interrupted by actuation of the actuator 17.

Referring now to FIG. 2, there is shown a perspective view of an alternate embodiment of the ultraviolet sterilization apparatus. In the illustrated embodiment, the housing 11 is spherical with a plurality of lights 14 disposed thereon. Each light 14 of the plurality of lights is circular, such that beams of light are released from the housing 11 in every direction. In the illustrated embodiment, a plurality of apertures is disposed in the housing 11, such that each light 14 of the plurality of lights extends through the housing 11. Furthermore, in the illustrated embodiment each light 14 of the plurality of lights is spaced equidistantly from the other lights of the plurality of lights. In one embodiment, the housing 11 is freely rotatable, such that the plurality of lights 14 can be adjusted to any desired location regardless of interference of portions of the housing 11 disposed between each light 14 of the plurality of lights.

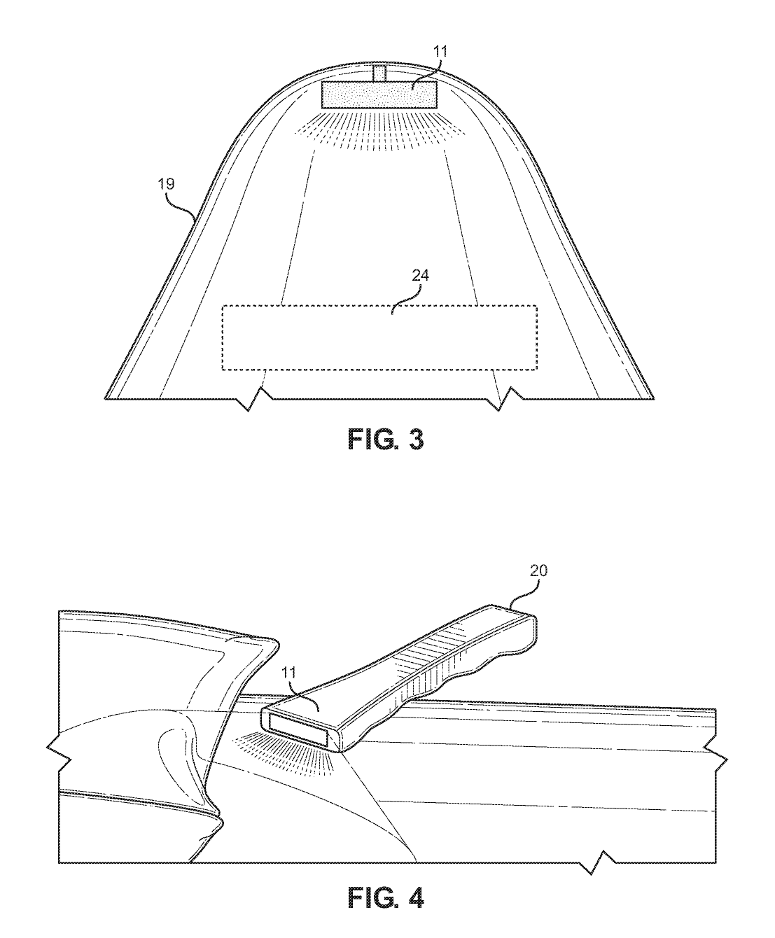

Referring now to FIG. 3, there is shown a perspective view of an alternate embodiment of the ultraviolet sterilization apparatus. In the illustrated embodiment, the housing 11 is disposed on a ceiling of a tent 19. The tent 19 is configured to be collapsible around the housing 11, such that the ultraviolet sterilization apparatus can be easily transported and can be stored without taking up as much room.

Furthermore, the tent 19 prevents dilution of the ultraviolet light produced by the lights of the ultraviolet sterilization apparatus. The tent 19 is of any size suitable for sanitizing a targeted object. For example, the tent 19 can be of a smaller size, such that an object such as a payment terminal can be sanitized. Additionally, the tent 19 can be of a larger size, such that an area, such as a surgical site, can be sanitized.

Referring now to FIG. 4, there is shown a perspective view of an alternate embodiment of the ultraviolet sterilization apparatus. In the illustrated embodiment, the housing 11 comprises a handle 20. By providing a handle, the ultraviolet sterilization apparatus is able to be used in sanitizing large objects, such as beds or other seating surfaces, while also being portable. The handle 20 can provide convenience to a user when sanitizing an object having overhands that would not be accessible to the ultraviolet radiation via an overhead or stationary housing.

In the illustrated embodiment, the handle 20 is ergonomic, such that the handle can be more effectively grasped by the user. In the illustrated embodiment, the handle is ergonomic via a plurality of protrusions extending outward from a bottom surface of the handle 20. As such, the user may rest their fingers between the protrusions, such that the handle 20 can be comfortably grasped. In an additional embodiment, the handle 20 may comprises a material having an increased friction coefficient thereon. As such, the amount of grip provided to the user is increased, decreasing the risk of the ultraviolet sterilization apparatus being dropped and damaged.

Furthermore, in the illustrated embodiment, the housing 11 and the handle 20 form a single unitary piece. As such, the ultraviolet sterilization apparatus is more stream-lined and easier to use. In other embodiments, the handle 20 and the housing 11 are freely separable from each other, such that the housing 11 can be used with other handles 20 or mechanisms for holding the housing 11. For example a longer handle 20 may be desired for certain uses of the ultraviolet sterilization apparatus.

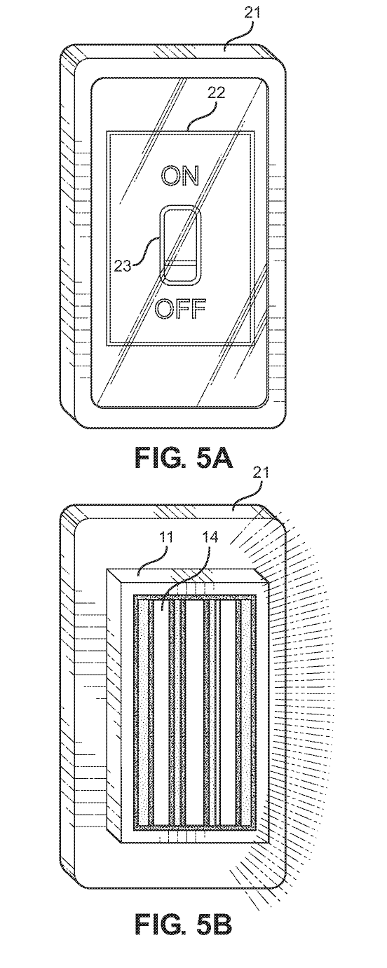

Referring now to FIGS. 5A and 5B, there are shown a front perspective view and a rear perspective view of an alternate embodiment of the ultraviolet sterilization apparatus respectively. In the illustrated embodiment, the housing 11 is integrated upon a mobile device 21. The housing 11 is in operable connection with the mobile device 21. In the illustrated embodiment, the mobile device comprises an application. The application is configured to activate and deactivate the light 14. The application comprises a graphical user interface 22, or GUI. The graphical user interlace 22 comprises a digital actuator 23. The digital actuator 23 is configured to activate and deactivate the light 14.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1) An ultraviolet sterilization apparatus, comprising:
a housing having a base disposed oppositely a top with at least one sidewall disposed therebetween;
a light disposed on an external surface of the housing;
the light configured to emit ultraviolet radiation;
the light in operable connection with an actuator.

2) The ultraviolet sterilization apparatus of claim 1, wherein the housing is disposed on a ceiling of a tent.

3) The ultraviolet sterilization apparatus of claim 2, herein the tent is collapsible.

4) The ultraviolet sterilization apparatus of claim 1, wherein the housing comprises a handle.

5) The ultraviolet sterilization apparatus of claim 1, wherein the housing is integrated upon a mobile device and is in operable connection with the mobile device.

6) The ultraviolet sterilization apparatus of claim 5, wherein the mobile device comprises an application configured to activate and deactivate the light.

7) The ultraviolet sterilization apparatus of claim 1, wherein the housing is spherical and comprises a plurality of apertures evenly disposed around a surface area of the spherical housing.
